(19) United States
(12) Patent Application Publication
(10) Pub. No.: US 2023/0337975 A1
(43) Pub. Date: Oct. 26, 2023

(54) SYSTEMS AND METHODS FOR NEUROLOGICAL REHABILITATION USING VIRTUAL REALITY

(71) Applicant: Somos Inc., Oregon City, OR (US)

(72) Inventors: Peter Lund, Clackamas, OR (US); Julius Jockusch, Oregon City, OR (US); Albert Chi, Oregon City, OR (US)

(21) Appl. No.: 18/304,722

(22) Filed: Apr. 21, 2023

Related U.S. Application Data
(60) Provisional application No. 63/363,372, filed on Apr. 21, 2022.

(51) Int. Cl. A61B 5/00; A61B 5/397

(52) U.S. Cl. CPC A61B 5/486; A61B 5/397; A61B 5/745; A61B 5/7267; A61B 2505/09; G06F 3/015

(57) ABSTRACT

A method can include: acquiring via one or more electromyography (EMG) sensors in electrical contact with a patient, one or more electrical signals; mapping the one or more electrical signals to one or more intended movements via an EMG signal classifier; applying the one or more intended movements to a simulated body region; and rendering a movement of the simulated body region using a virtual reality (VR) display device.

### CROSS-REFERENCE TO RELATED APPLICATION

This application claims priority to and the benefit of U.S. Provisional Application No. 63/363,372, filed on Apr. 21, 2022 and titled SYSTEMS AND METHODS FOR PARALYSIS REHABILITATION USING VIRTUAL REALITY, the full content of which is hereby incorporated herein by reference in its entirety.

### TECHNICAL FIELD

Embodiments of the subject matter disclosed herein relate to using virtual reality or augmented reality simulation of body movement to improve mobility in paralyzed or mobility limited body regions.

### BACKGROUND

Paralysis or impaired mobility from neurological injury or disease in one or more body regions, e.g., arms, legs, neck, etc. affects millions of people. Paralysis may significantly impact a person's physical health, independence, and overall quality of life. Causes of paralysis may include neuromuscular diseases, spinal cord injuries, and stroke. Conventional approaches to rehabilitate paralysis include passive exercises, electrical stimulation, electroacupuncture, and mental training (e.g., visualization of movement of the affected body region). Although conventional rehabilitation approaches may improve mobility of the affected body region in some cases, a lack of visual muscular response in the affected areas may lead to a reduction in both compliance and effort displayed during the rehabilitation process and after. Thus, there remains impetus to find more effective approaches for regaining movement in paralyzed or mobility limited body regions.

### SUMMARY

The inventors herein have developed systems and methods which have demonstrated unexpected efficacy in stimulating adaptive neuroplastic response in patients with paralysis or limited mobility in one or more body regions. In one example, a system for neurological rehabilitation of a body region comprises: an electromyography (EMG) sensor, a virtual reality (VR) display device, a non-transitory memory (wherein the non-transitory memory includes an EMG signal classifier, and instructions), and a processor (wherein the processor is communicatively coupled to the EMG sensor, the VR display device, and the non-transitory memory), and wherein, when executing the instructions, the processor is configured to initialize a VR environment, acquire one or more EMG signals via the EMG sensor, wherein the EMG sensor is in electrical contact with the body region of a user, map the one or more EMG signals into one or more intended movements of the body region, apply the one or more intended movements to a simulated body region, wherein the simulated body region corresponds to the body region, apply virtual physics to the simulated body region, render, in real time, a movement of the simulated body region based on the one or more intended movements and the virtual physics, and update a state of the virtual environment based on the movement of the simulated body region.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 is a first block diagram of an exemplary system for acquiring EMG signals from a body region, and rendering movement of a simulation of the body region in a virtual environment based on the EMG signals;

FIG. 2 is a flow chart of a method for simulating body region movement of paralyzed or mobility limited body regions, based one or more EMG signals;

FIG. 3 is a flow chart of a method for generating a map between EMG signals and intended movements of a body region;

FIG. 4 is an exemplary depiction of a simulated body region in a virtual environment engaging in a rehabilitation game;

FIG. 5 is an exemplary depiction of a simulated body region in a virtual environment engaging in a rehabilitation game; and

FIG. 6 is a second block diagram of an exemplary system for acquiring signals from a body region, and rendering movement of a simulation of the body region in a virtual environment based on the EMG signals.

The drawings illustrate specific aspects of the described system and methods for neurological rehabilitation using virtual reality simulation of body region movement. Together with the following description, the drawings demonstrate and explain the structures, methods, and principles described herein. In the drawings, the size of components may be exaggerated or otherwise modified for clarity. Well-known structures, materials, or operations are not shown or described in detail to avoid obscuring aspects of the described components, systems and methods.

### DETAILED DESCRIPTION

The current disclosure describes systems and methods which may facilitate advantageous neuroplastic adaptations in patients with paralysis or limited mobility resulting from neurological injury or disease in one or more body regions. In one example, the current disclosure teaches placing electromyography (EMG) electrodes on pre-determined muscle groups in, or around, the body region. The EMG electrodes are configured to pick up faint electrical signals from the pre-determined muscle groups, and the faint electrical signals may be classified into one or more intended movements. As used herein, an intended movement refers to a movement corresponding to a muscle activation, which may or may not translate into a physical movement of the body region, i.e., the muscle activation may be insufficient to trigger muscle contraction of great enough magnitude to enable physical movement of the body region. The intended movements may then be translated into simulated movement of a virtual simulacrum of the body region, rendered in a virtual environment by a virtual reality (VR) display device. The patient is motivated to move the body region to interact with the virtual environment, e.g., completing tasks, playing games. The inventors herein have discovered that by submersing the mind of the patient in a virtual environment, wherein intended movement of a paralyzed or mobility limited body region translates into simulated movement of a virtual representation of the body region, the body of the patient responds via a process whereby connections between the nervous system and the muscles of the body region are strengthened and/or coordinated, such that conscious mobility of the body region may be increased.

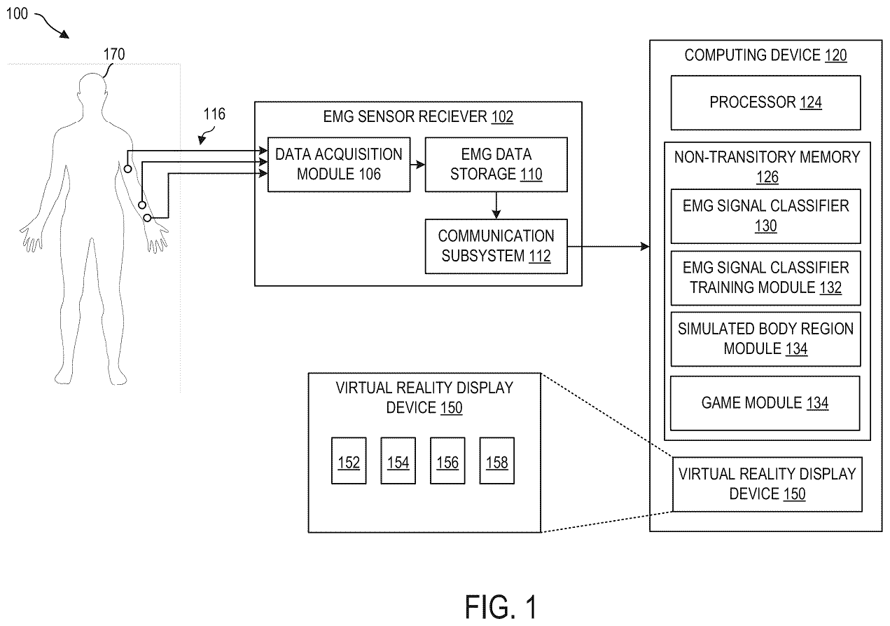

Referring to FIG. 1, one embodiment of a VR rehabilitation system 100 is shown. VR rehabilitation system 100 comprises an EMG sensor receiver 102, and a computing device 120 communicably coupled thereto. The EMG sensor receiver 102 is configured to receive electrical signals (also referred to herein as EMG sensor signals) from a plurality of EMG sensors 116, coupled to one or more body regions of patient 170, and relay the electrical signals to computing device 120. The electrical signals measured by EMG sensors 116 correspond to electrical activity of one or more muscle groups proximal to each respective EMG sensor.

The EMG signals measured by EMG sensor receiver 102 comprise time series data, wherein an electrical potential (voltage) between two or more electrodes of the plurality of EMG sensors 116, in electrical contact with patient 170's skin, is recorded as a function of time. The EMG signal data acquired by EMG sensor receiver 102 may be transferred to computing device 120, via communication subsystem 112, for processing and classification into one or more intended movements.

EMG sensor receiver 102 includes a plurality of EMG sensors 116, which, in the example shown in FIG. 1, are placed on a left arm of patient 170. The EMG sensors 116 may include one or more electrodes, configured to measure a difference in electrical potential between positions of two or more electrodes of EMG sensors 116. The current disclosure also provides for EMG sensor placement other than that described above with reference to EMG sensors 116.

EMG sensors 116 may be electrically coupled to data acquisition module 106 of EMG sensor receiver 102. Data acquisition module 106 is configured to measure electrical potential differences between two or more electrodes of EMG sensors 116 as a function of time. In some embodiments, the EMG signals acquired by EMG sensors 116 may be stored in EMG data storage 110. In some embodiments, data acquisition module 106 may be configured to receive analog electrical signals from EMG sensors 116, amplify and/or filter the analog signals, and convert the analog signals to digital signals, before transmitting the digital signals to computing device 120. In another embodiment, data acquisition module 106 may convert the analog electrical signals from EMG sensors 116 to a digital signal, and may amplify and/or filter the digital signal before transmitting the digital signal to computing device 120. In some embodiments, data acquisition module 106 may be configured to differential amplify signals from each EMG sensor, thereby adjusting for differences in signal intensity.

Data acquisition module 106 is communicably coupled with EMG data storage 110, and may write EMG data acquired from patient 170 to EMG data storage 110 for storage. EMG data storage 110 may comprise non-transitory memory, wherein the EMG data acquired by data acquisition module 106 may be stored. EMG data stored in EMG data storage 110 may comprise time series data, wherein an amplitude of the electrical potential difference between two or more electrodes in electrical contact with patient 170 is recorded at regular intervals in time, wherein each recorded electrical potential difference is time stamped with the time of acquisition, thereby creating time series data. In some embodiments, EMG data storage 110 may comprise a memory card, a flash drive, or a removable hard drive. In some embodiments, EMG data storage 110 may be integrated into EMG sensor receiver 102, and may include a solid state drive (SSD), hard disk drive (HDD).

In some embodiments, EMG sensor receiver 102 and computing device 120 may be communicably coupled by communication subsystem 112. In one embodiment, communication subsystem 112 may comprise a wireless or wired connection configured to transfer EMG data from EMG data storage 110 of EMG sensor receiver 102 to computing device 120. In some embodiments, communication subsystem 112 may enable EMG sensor receiver 102 and EMG processing device to be in substantially continuous communicative coupling, via a wireless network, enabling computing device 120 to receive real time EMG data from EMG sensor receiver 102.

Communication subsystem 112 may include wired and/or wireless communication devices compatible with one or more different communication protocols. As non-limiting examples, communication subsystem 112 may be configured to transfer EMG data from EMG data storage 110 to computing device 120 via a wireless telephone network, a wireless local area network, a wired local area network, a wireless wide area network, a wired wide area network, etc. In some embodiments, communication subsystem 112 may allow EMG sensor receiver 102 to send and/or receive data to and/or from other devices via a network such as the public Internet. For example, communication subsystem 112 may communicatively couple EMG sensor receiver 102 with consumer Computing device 120 via a network, such as the public Internet.

EMG data acquired by EMG sensor receiver 102 may be transferred to computing device 120 for processing (e.g., signal filtering, normalization, noise suppression, etc.), classification into one or more intended movements, and rendering/display of the one or more intended movements in a virtual environment.

Computing device 120 includes a processor 124 configured to execute machine readable instructions stored in non-transitory memory 126. Processor 124 may be single core or multi-core, and the programs executed thereon may be configured for parallel or distributed processing. In some embodiments, the processor 124 may optionally include individual components that are distributed throughout two or more devices, which may be remotely located and/or configured for coordinated processing. In some embodiments, one or more aspects of the processor 124 may be virtualized and executed by remotely-accessible networked computing devices configured in a cloud computing configuration.

Non-transitory memory 126 may store EMG signal classifier 130, EMG signal classifier training module 132, simulated body region module 134, game module 134, and instructions for execution one or more of the operations of one or more of the methods described herein. In some embodiments, the non-transitory memory 126 may include components disposed at two or more devices, which may be remotely located and/or configured for coordinated processing. In some embodiments, one or more aspects of the non-transitory memory 106 may include remotely-accessible networked storage devices configured in a cloud computing configuration.

EMG signal classifier 130 is configured to receive one or more EMG signals, from one or more of the plurality of EMG sensors 116, and map (or classify) the EMG signals to one or more intended movements of the muscle groups associated with the EMG sensors 116. In some embodiments, EMG signal classifier 130 includes a linear regression model, wherein received EMG signals are linearly correlated to a set of pre-determined movements based on one or more adjustable parameters. In some embodiments, the EMG signal classifier 130 may include one or more machine learning models, including but not limited to, neural networks, deep neural networks, clustering models (e.g., k-means, DBSCAN, affinity propagation, etc.).

EMG signal classifier training module 132 is configured to train, or calibrate, one or more models/maps stored in EMG signal classifier 130. In some embodiments, EMG signal classifier training module 132 may include instructions for determining one or more linear model parameters based on EMG signals received during a calibration process, such as the calibration process described in method 300, below. In some embodiments, EMG signal classifier training module 132 may include instructions for performing an unsupervised learning routine, such as clustering EMG signal data into groups corresponding to one or more intended movements. In some embodiments, EMG signal classifier training module 132 may include instructions for performing one or more supervised learning routines, such as a gradient descent algorithm wherein a neural network or deep neural network may learn a mapping from an EMG signal space to an intended movement classification.

Simulated body region module 134 is configured to model a virtual representation of one or more body regions of a user. In some embodiments, simulated body region module 134 may include a model of a body region, including simulated points of articulation, and muscles, wherein the muscles may correspond to one or more muscles of patient 170 proximal to one or more of EMG sensors 116. In one example, simulated body region module 134 may include a model of a right arm, including a simulated elbow joint, wrist joint, one or more simulated bones (e.g., radius, ulna, humerous, etc.), and one or more simulated muscles (triceps brachii, biceps brachii, brachialis, brachioradialis, etc.). Simulated body region module 134 may include instructions for translating one or more intended movements identified by EMG signal classifier 130 into positional adjustments of one or more points of a simulated body region relative to a virtual coordinate system.

Game module 134 is configured with instructions for rendering one or more virtual environments, and enabling a user to move through and interact with the virtual environment according to one or more game rules. In some embodiments, game module 134 may include separate game modules for implementing one or more games in one or more virtual environments. In one example, game module 134 may include instructions for implementing Apple Grab: a virtual environment wherein the user may “walk” around and virtually interact with objects on several tables, includes the ability to pick up objects, stack objects, and throw objects. In another example game module 134 may include instructions for implementing Target Shoot: a target shooting game wherein the user uses a virtual hand to aim at and shoot targets as the targets move through the virtual environment. In another example, game module 134 may include instructions for implementing Crystal Break: a target breaking game where the user uses a virtual hand to aim at and break targets as they move through the virtual environment.

VR display device 150 may include one or more displays, such as display 152, display 154, display 156, and display 158. Displays 152-158 may enable an immersive visual display, such that a user may be “surrounded” by displays 152-158. In some embodiments, VR display device comprises glasses, or goggles, which may be worn over the eyes of a user, such that while wearing VR display device 150, a user's view may consist primarily, or entirely, of one or more of displays 152-158.

It should be understood that VR rehabilitation system 100, shown in FIG. 1, is for illustration, not for limitation. Another appropriate VR rehabilitation system may include more, fewer, or different components.

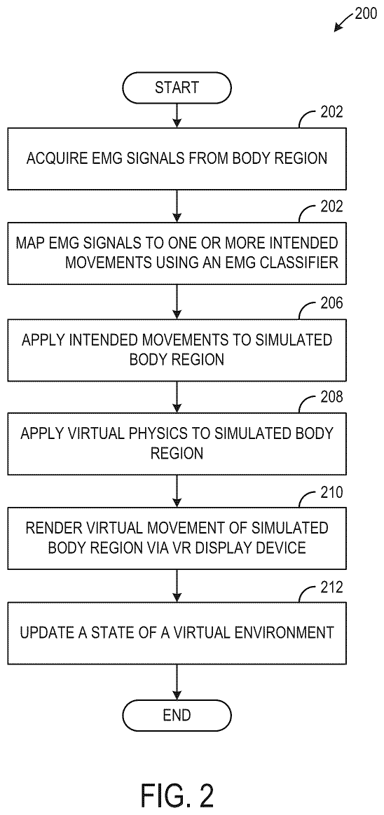

Referring to FIG. 2, an exemplary method 200 for simulating movement of a body region of a user in a virtual environment using a VR rehabilitation system, is shown. In some embodiments, method 200 may be executed by a VR rehabilitation system, such as VR rehabilitation system 100 shown in FIG. 1, based on instructions stored in non-transitory memory 126. In one example, method 200 may be executed as part of a rehabilitation regimen.

Method 200 begins at operation 202, wherein the VR rehabilitation system acquires EMG signals from a body region of the user. The VR rehabilitation system may acquire one or more EMG signals, comprising electrical signals, from one or more EMG sensors in electrical contact with the body region of the patient. The one or more EMG sensors may be placed proximal to one or more pre-determined muscle groups, in, or around, the body region. In one example one or more sensors may be placed near the biceps brachii to measure electrical signals corresponding to flexion and one or more sensors may be placed near the triceps brachii for acquiring electrical signals corresponding to extension of the forearm.

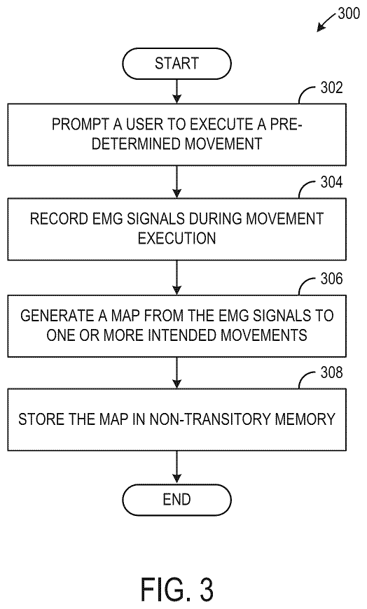

At operation 204, the VR rehabilitation system maps EMG signals to one or more intended movements using an EMG signal classifier. In one example, EMG signals may be correlated to one or more intended movements using a linear regression model, wherein a series of EMG samples with intended movement labels are used to train the linear regression model during the calibration process, such as described below with reference to method 300, shown in FIG. 3.

At operation 206, the VR rehabilitation system applies the intended movements to a simulated body region. The simulated body region corresponds to the body region (that is, the physical body region). In one example, the body region may comprise a right arm of the patient, and the virtual body region may comprise a virtual representation or simulation of a right arm of the patient. In some embodiments, applying the intended movement to the simulated body region may include translating the one or more intended movements into positional transformations of a model of the body region, relative to a coordinate system of the virtual environment.

At operation 208, the VR rehabilitation system applies virtual physics to the simulated body region. In some embodiments, to provide a more engaging simulation, one or more physical interactions between the simulated body region and the virtual environment may be performed. In one example, lighting of a body region (e.g., shading, reflection, etc.) may be determined based on a position of the simulated body region with respect to one or more simulated light sources. In another example, a velocity of the simulated body region may be adjusted based on a virtual viscosity of one or more fluid mediums through which the simulated body regions is moving.

At operation 210, the VR rehabilitation system renders virtual movement of the simulated body region via a VR display device. In some embodiments, rendering the virtual movement of the simulated body region may include displaying a sequence of frames via the VR display device of the body region moving through a plurality of positions while undergoing the virtual movement.

At operation 212, the VR rehabilitation system updates a state of the virtual environment based on the virtual movement of the simulated body region. In some embodiments, operation 212 includes rendering one or more interactions of the body region with the virtual environment e.g., breaking an item, shooting a gun, adjusting the position of an item, and so on, according to the logic of the currently implemented game, as well as updating a state of the game, e.g., updating a score, establishing that a win condition has been met, initiating a chain of events caused by the virtual movement of the body region, etc.

Following operation 212, method 200 may end. It will be appreciated that method 200 may be executed repeatedly to enable a user to perform a plurality of simulated movements. In some embodiments, multiple instances of method 200 may be executed in parallel, thereby enabling rendering of one or more movements of the simulated body region in real time.

Referring to FIG. 3, an exemplary method 300 for learning a map from EMG signals to intended movements, is shown. Method 300 may be executed by a VR rehabilitation system, such as VR rehabilitation system 100, shown in FIG. 1. In some embodiments, method 300 may be executed prior to initialization of a VR rehabilitation session. In other words, a map may be re-learned/re-calibrated each time a user begins a session on the VR rehabilitation system, thereby increasing correlation between EMG signals and intended movements of the user.

Method 300 starts at operation 302, wherein the VR rehabilitation system prompts a user, via a visual indication in a VR display device, to perform one or more pre-determined movements (e.g., to press a virtual button displayed by the VR display device). In one example, a pre-determined movement may include extending an arm, pronating a hand, etc.

At operation 304, the VR rehabilitation system records EMG signals during movement execution. The VR rehabilitation system may measure EMG signals as described in more detail above, with reference to FIG. 1.

At operation 306, the VR rehabilitation system generates a map from EMG signals to one or more intended movements. In some embodiments, at operation 306, the VR rehabilitation system determines one or more model parameters of a linear model by fitting said linear model to the one or more EMG signals recorded at operation 304. In some embodiments, the one or more EMG signals recorded at operation 304 may be labeled based on the pre-determined movement prompted at operation 302, wherein the recorded EMG signal and the movement label may be used as a training data pair in a supervised training routine of a machine learning model. In some embodiments, the VR rehabilitation system may store the one or more recorded EMG signals in a database or other memory structure, and may map newly acquired EMG signals to intended movements by comparing similarity of newly acquired EMG signals to previously index EMG signals (e.g., via an approximate nearest neighbors algorithm).

At operation 308, the VR rehabilitation system, store the map in non-transitory memory. In some embodiments, the map may comprise a trained machine learning model, wherein the VR rehabilitation system may store the trained parameters of the machine learning model. In some embodiments, at operation 308, the VR rehabilitation system may store one or more linear model parameters determined based on the EMG signals received at operation 304. In some embodiments, the VR rehabilitation system stores the EMG signal data (or an encoding based on the EMG signal data) acquired at operation 304, along with labels of the intended movement associated with each EMG signal (or cluster).

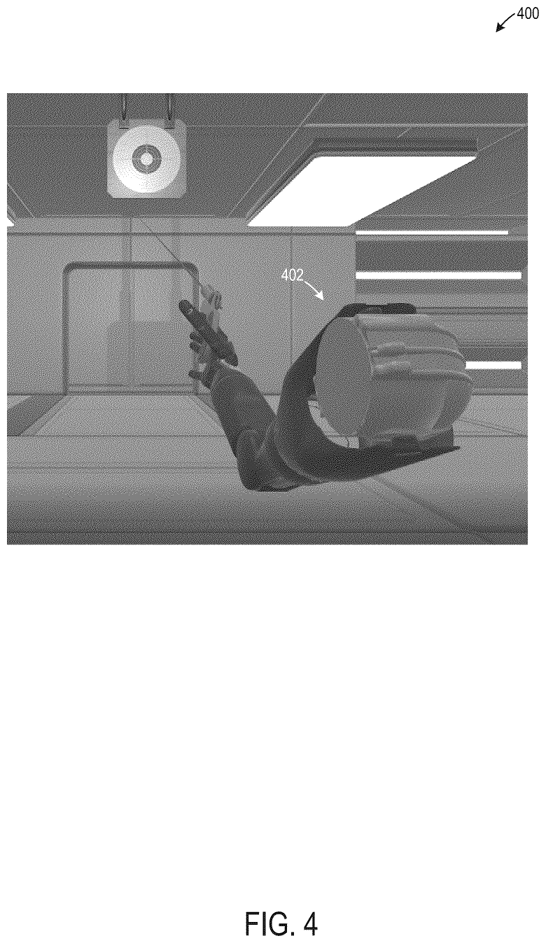

Referring to FIG. 4, an example of a simulated body region 402 is shown rendered in a virtual environment 400.

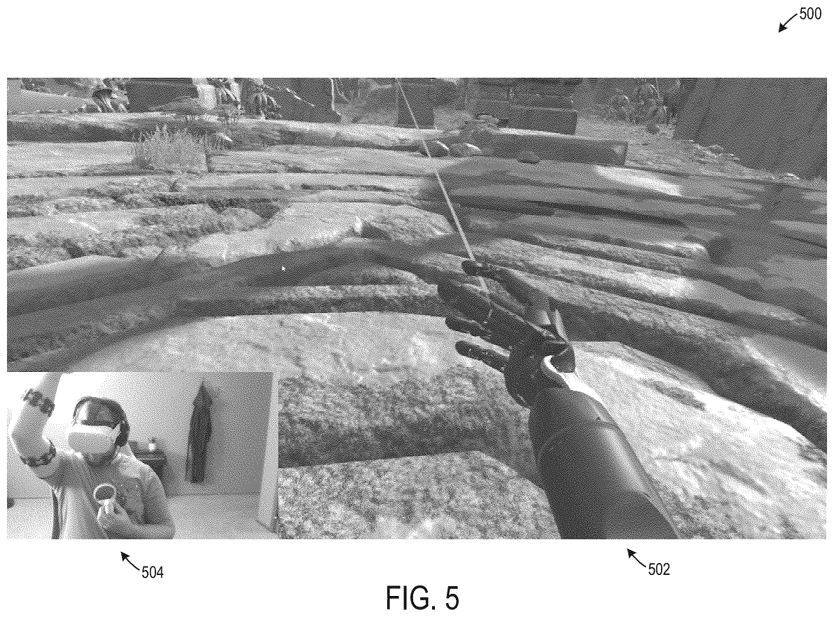

Referring to FIG. 5, an example of a simulated body region 502 is shown rendered in a virtual environment 500, with an image of a user 504 wearing a plurality of EMG sensors configured to control position of body region 502 relative to a coordinate system of virtual environment 500.

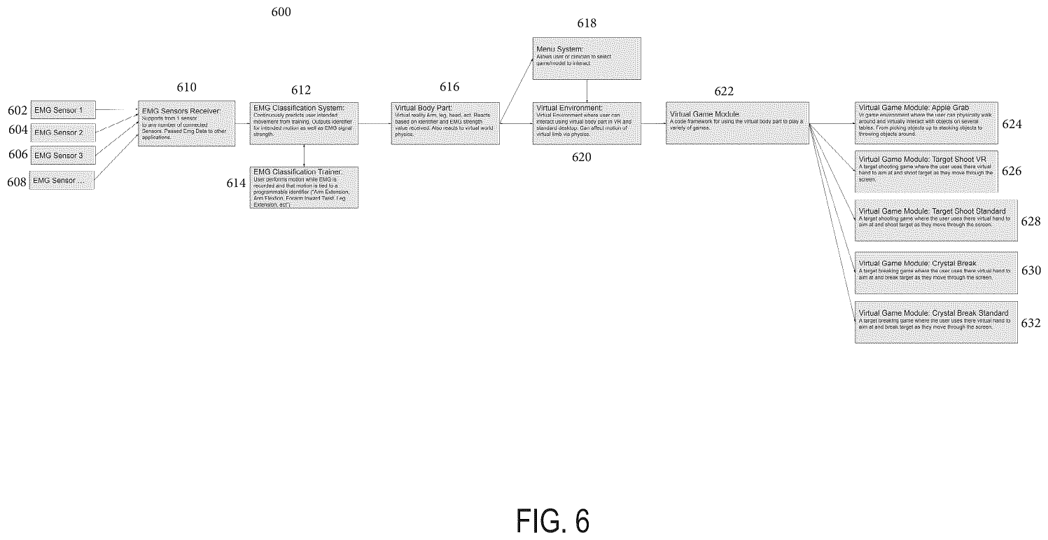

Referring to FIG. 6, a second block diagram illustrating a VR rehabilitation system 600 is shown. In the example, the system 600 includes multiple EMG sensors 602, 604, 606, 608 and an EMG sensor receiver 610 configured to receive EMG data from the EMG sensors 602, 604, 606, 608 and pass the EMG data to other applications. The system 600 also includes an EMG classification system 612 configured to continuously predict user intended movement from training and output an identifier for intended motion as well as EMG signal strength. The system 600 also includes an EMG classification trainer 614 wherein a user performs motion while the EMG is being recorded and such motion is tied to a programmable identifier.

In the example, a virtual body part 616 can react based on the identifier and EMG strength value received, and can also react to virtual world physics. The system 600 also includes a menu system 618 configured to allow a user or clinician to select a game or model for interaction. The system 600 also includes a virtual environment 620 where a user can interact using the virtual body part in VR and standard desktop. The system 600 also includes a virtual game module 622 providing a code framework for using the virtual body part to play a variety of games.

The games associated with the virtual game module 622 can include: Apple Grab 624 (e.g., a virtual game environment where a user can physically walk around and virtually interact with objects on several tables), Target Shoot VR 626 and Target Shoot Standard 628 (e.g., a target shooting game where a user can use their virtual hand to aim at and shoot a target as they move through the screen), and Crystal Break VR 630 and Crystal Break Standard 632 (e.g., a target breaking game where a user can use their virtual hand to aim at and break a target as they move through the screen).

Aspects of the disclosure may operate on particularly created hardware, firmware, digital signal processors, or on a specially programmed computer including a processor operating according to programmed instructions. The terms controller or processor as used herein are intended to include microprocessors, microcomputers, Application Specific Integrated Circuits (ASICs), and dedicated hardware controllers.

One or more aspects of the disclosure may be embodied in computer-usable data and computer-executable instructions, such as in one or more program modules, executed by one or more computers (including monitoring modules), or other devices. Generally, program modules include routines, programs, objects, components, data structures, and so on, that perform particular tasks or implement particular abstract data types when executed by a processor in a computer or other device. The computer executable instructions may be stored on a computer readable storage medium such as a hard disk, optical disk, removable storage media, solid state memory, Random Access Memory (RAM), etc. As will be appreciated by one of skill in the art, the functionality of the program modules may be combined or distributed as desired in various aspects. In addition, the functionality may be embodied in whole or in part in firmware or hardware equivalents such as integrated circuits, FPGAs, and the like.

Particular data structures may be used to more effectively implement one or more aspects of the disclosure, and such data structures are contemplated within the scope of computer executable instructions and computer-usable data described herein.

The disclosed aspects may be implemented, in some cases, in hardware, firmware, software, or any combination thereof. The disclosed aspects may also be implemented as instructions carried by or stored on one or more or computer-readable storage media, which may be read and executed by one or more processors. Such instructions may be referred to as a computer program product. Computer-readable media, as discussed herein, means any media that can be accessed by a computing device. By way of example, and not limitation, computer-readable media may comprise computer storage media and communication media.

Computer storage media means any medium that can be used to store computer-readable information. By way of example, and not limitation, computer storage media may include RAM, ROM, Electrically Erasable Programmable Read-Only Memory (EEPROM), flash memory or other memory technology, Compact Disc Read Only Memory (CD-ROM), Digital Video Disc (DVD), or other optical disk storage, magnetic cassettes, magnetic tape, magnetic disk storage or other magnetic storage devices, and any other volatile or nonvolatile, removable or non-removable media implemented in any technology. Computer storage media excludes signals per se and transitory forms of signal transmission.

Communication media means any media that can be used for the communication of computer-readable information. By way of example, and not limitation, communication media may include coaxial cables, fiber-optic cables, air, or any other media suitable for the communication of electrical, optical, Radio Frequency (RF), infrared, acoustic or other types of signals.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method comprising:
acquiring via one or more electromyography (EMG) sensors in electrical contact with a patient, one or more electrical signals;
mapping the one or more electrical signals to one or more intended movements via an EMG signal classifier;
applying the one or more intended movements to a simulated body region; and
rendering a movement of the simulated body region using a virtual reality (VR) display device.

2. The method of claim 1, wherein the one or more intended movements include a unique identifier, and a strength of an electrical signal associated with the intended movement.

3. The method of claim 1, wherein the EMG signal classifier comprises a trained linear regression model.

4. The method of claim 1, wherein the one or more EMG sensors are in electrical contact with a body region of the patient, wherein the body region is paralyzed and/or displays reduced mobility from neurological injury or disease, and wherein the body region corresponds to the simulated body region.

5. The method of claim 1, the method further comprising:
updating a state of a virtual environment based on the movement of the simulated body region.

6. The method of claim 1, the method further comprising:
applying virtual physics to the simulated body region; and
incorporating the virtual physics in rendering the movement of the simulated body region.

7. A method comprising:
prompting a user to execute a pre-determined movement;
recording electrical signals received from one or more electromyography (EMG) sensors in electrical contact with a body region of the user;
generating a map from the electrical signals to one or more intended movements of the body region of the user; and
storing the map in a non-transitory memory.

8. The method of claim 7, wherein the map comprises a linear regression model.

9. The method of claim 7, wherein the map comprises a machine learning model.

10. The method of claim 7, the method further comprising:
rendering in real time the one or more intended movements via a virtual reality display device.

11. A system comprising:
an electromyography (EMG) sensor;
a virtual reality (VR) display device;
a non-transitory memory, wherein the non-transitory memory includes an EMG signal classifier, and instructions; and
a processor, wherein the processor is communicatively coupled to the EMG sensor, the VR display device, and the non-transitory memory, and wherein, when executing the instructions, the processor is configured to:
initialize a VR environment;
acquire one or more EMG signals via the EMG sensor, wherein the EMG sensor is in electrical contact with a body region of a user;
map the one or more EMG signals into one or more intended movements of the body region;
apply the one or more intended movements to a simulated body region, wherein the simulated body region corresponds to the body region;
apply virtual physics to the simulated body region;
render, in real time, a movement of the simulated body region based on the one or more intended movements and the virtual physics; and
update a state of the virtual environment based on the movement of the simulated body region.

12. The system of claim 11, wherein the one or more intended movements include a unique identifier, and a strength of an electrical signal associated with the intended movement.

13. The system of claim 11, wherein the EMG signal classifier comprises a trained linear regression model.

14. The system of claim 11, wherein the body region is paralyzed and/or displays reduced mobility from neurological injury or disease, and wherein the body region corresponds to the simulated body region.

15. The system of claim 11, wherein the processor is further configured to update a state of a virtual environment based on the movement of the simulated body region.

16. The system of claim 11, wherein the processor is further configured to:
apply virtual physics to the simulated body region; and
incorporate the virtual physics in rendering the movement of the simulated body region.

17. One or more tangible, non-transitory computer-readable media storing executable instructions that, when executed by a processor, cause the processor to perform the method of claim 1.

18. One or more tangible, non-transitory computer-readable media storing executable instructions that, when executed by a processor, cause the processor to perform the method of claim 7.

19. The method of claim 7, wherein the body region is paralyzed and/or displays reduced mobility from neurological injury or disease.

20. The system of claim 11, wherein the non-transitory memory is configured to store the state of the virtual environment.
